(19) United States
(12) Patent Application Publication
(10) Pub. No.: US 2021/0193285 A1
(43) Pub. Date: Jun. 24, 2021

(54) A SYSTEM AND METHOD FOR USE IN DISEASE TREATMENT MANAGEMENT

(71) Applicant: Dreamed Diabetes Ltd., Petach Tikvah (IL)

(72) Inventors: Revital Nimri, Petach Tiqwa (IL); Moshe Phillip, Givatayim (IL); Eran Atlas, Petach Tiqwa (IL); Ido Muller, Ness Tsiona (IL); Ilana Klovatch, Petach Tiqwa (IL); Eran Agmon, Ra'anana (IL)

(21) Appl. No.: 16/756,190

(22) PCT Filed: Oct. 16, 2018
(86) PCT No.: PCT/IB2018/058001

§ 371 Date: Apr. 15, 2020

Related U.S. Application Data
(60) Provisional application No. 62/574,253, filed on Oct. 19, 2017.

(51) Int. Cl. G16H 20/17; G16H 40/67; G16H 50/30; G16H 50/70; G16H 20/60; G16H 40/40; G16H 10/60; G16H 40/20; A61M 5/172

(52) U.S. Cl. CPC G16H 20/17; G16H 40/67; G16H 50/30; G16H 50/70; G16H 20/60; G16H 40/40; A61M 2205/52; G16H 40/20; A61M 5/1723; A61M 2205/50; A61M 2230/201; A61M 2230/63; A61M 2205/3303; G16H 10/60

(57) ABSTRACT

Aspects of embodiments pertain to a method for use in disease treatment management comprising: receiving data indicative of pump treatment parameters; analyzing physiological data during use of a pump; said physiological data being indicative of a physiological characteristic of the patient; analyzing the received pump treatment parameters data to thereby identify at least one patient-related treatment characteristic; creating data indicative of multiple daily injections (MDI) treatment parameters by automatically determining individualized insulin dosing injection parameters data based on said at least one patient-related treatment characteristic and said physiological data.

### TECHNOLOGICAL FIELD

The present invention relates to a system and method for use in disease treatment management and more specifically for diabetes care.

### BACKGROUND ART

References considered to be relevant as background to the presently disclosed subject matter are listed below:
1. US 2013/0324824.
2. US 2016/0117481.
3. US 2017/0189614.
4. US 2017/0203037.
5. US 2017/0053101.
6. Miller K M, Foster N C, Beck R W, et al. Current State of Type 1 Diabetes Treatment in the U.S.: Updated Data From the T1D Exchange Clinic Registry. Diabetes Care. 2015; 38(6):971-978.
7. NH C. IDF Diabetes Atlas. Seventh Edition ed. 2015, Page 16.
8. McKnight J A, Wild S H, Lamb M I, et al. Glycaemic control of Type 1 diabetes in clinical practice early in the 21st century: an international comparison. Diabet Med. 2014, pages 1036-50.
9. Heinemann L, Fleming G A, Petrie J R, Holl R W, Bergenstal R M, Peters A L. Insulin pump risks and benefits: a clinical appraisal of pump safety standards, adverse event reporting, and research needs: a joint statement of the European Association for the Study of Diabetes and the American Diabetes Association Diabetes Technology Working Group. Diabetes Care. 2015; 38(4):716-722.
10. Chico A, Tundidor D, Jordana L, et al. Changes in Insulin Requirements From the Onset of Continuous Subcutaneous Insulin Infusion (CSII) Until Optimization of Glycemic Control. J Diabetes Sci Technol. 2014; 8(2):371-377.
11. Conrad S C, McGrath M T, Gitelman S E. Transition from multiple daily injections to continuous subcutaneous insulin infusion in type 1 diabetes mellitus. J Pediatr. 2002; 140(2):235-240.
12. Bachran R, Beyer P, Klinkert C, et al. Basal rates and circadian profiles in continuous subcutaneous insulin infusion (CSII) differ for preschool children, prepubertal children, adolescents and young adults. Pediatr Diabetes. 2012; 13(1):1-5.
13. Phillip M, Battelino T, Rodriguez H, et al. Use of insulin pump therapy in the pediatric age-group: consensus statement from the European Society for Paediatric Endocrinology, the Lawson Wilkins Pediatric Endocrine Society, and the International Society for Pediatric and Adolescent Diabetes, endorsed by the American Diabetes Association and the European Association for the Study of Diabetes. Diabetes Care. 2007; 30(6): 1653-1662.
14. Hofer S E, Heidtmann B, Raile K, et al. Discontinuation of insulin pump treatment in children, adolescents, and young adults. A multicenter analysis based on the DPV database in Germany and Austria. Pediatr Diabetes. 2010; 11(2):116-121.
15. Pickup J C, Yemane N, Brackenridge A, Pender S. Nonmetabolic complications of continuous subcutaneous insulin infusion: a patient survey. Diabetes Technol Ther. 2014; 16(3):145-149.
16. Guilhem I, Balkau B, Lecordier F, et al. Insulin pump failures are still frequent: a prospective study over 6 years from 2001 to 2007. Diabetologia. 2009; 52(12):2662-2664.
17. ADA—Standards of medical care in diabetes. Glycemic targets. Vol 41 (supplement 1); Diabetes Care 2018: S55-S64.

Acknowledgement of the above references herein is not to be inferred as meaning that these are in any way relevant to the patentability of the presently disclosed subject matter.

### BACKGROUND

The use of insulin pump therapy has become increasingly common among subjects with type 1 diabetes across all age groups. According to the Exchange T1 registry, more than 60% of type 1 diabetic patients use pump therapy and the number of users is anticipated to grow [6]. The worldwide incidence of Diabetes mellitus type 1 (T1DM) has risen dramatically over the past two decades especially among the young age group at a rate of 3% per year (65,000/year of newly diagnosed) [7]. Pump therapy has been gaining popularity as a treatment modality for patients with type 1 diabetes and recently in type 2 diabetes, and, as a result, the use of insulin pumps had greatly increased. In a recent update report from the T1D Exchange clinic registry, which included 16,061 adults and children with Type 1 diabetes, 60% used insulin pumps. The rate of use ranges from a low of 55% among subjects aged 18-25 years old to 65% among those aged 6-12 years old [6]. The rate of use appears to vary internationally and in Europe some centers reach rates of 70-93% [8]. The total number of pump users around the world is estimated at 0.75-1 million [9]. This number is anticipated to grow as technology becomes more available and becomes an integral part of daily life.

The introduction of new technologies such as software for big data collection, continuous glucose monitoring, smart pumps and glucometers and medical Apps and Software aims to provide patients with more tools to self-manage their disease, and healthcare professionals with the ability to better support and treat patients. Nevertheless, it requires the healthcare professionals to apply a new spectrum of theoretical knowledge and practical skills.

### General Description

Switching from multiple daily injections (MDI) to pump therapy is useful to a wide group of patients both with type 1 and type 2 diabetes. These may include patients who wish to optimize their glycemic control, newly diagnosed patients, during pregnancy, for transition to closed-loop therapy, and more. At times, the reverse transition from pump therapy to MDI is needed, such as during pump malfunction, or when a “pump holiday” is required. Subjects, who already use pumps, need from time to time to switch to MDI. This occurs frequently due to unexpected pump failure for a variety of reasons, which may be personal, a planned pump “vacation”, or due to other factors. However, patients are not prepared with the alternative basal and bolus dosing needed for MDI use. Furthermore, this can occur in times that no medical assistance is available and may endanger the patient with occurrence of ketosis. Sometimes, hospitalization is required for appropriately switching from one treatment modality to another.

The initiation of pump therapy is a complex process that involves a multidisciplinary team adequately trained, to address technical, clinical and psychological issues. A first step is to determine the initial pump settings. It should be noted that there are no clinically proven guidelines for the transfer of patients from one treatment modality to the other. In particular, there are no clear guidelines or recommendations available for pediatric care givers on how to facilitate the transition from MDI to pump therapy. The transition is mostly based on adults' empiric guidelines and prior insulin dosing that may not be optimal. Therefore, glycemic improvement after pump initiation may be delayed until further individual insulin dosing adjustments [10]. Furthermore, studies have showed that the basal rate may be different throughout the day and depends on the patients' age and pubertal stage [11,12]. Moreover, approaches vary between clinics and physicians. In most clinics, determination of initial settings is based on rules of thumb, empirical calculations, trial and error until eventual achieving of glycemic control. This process is time consuming requiring significant effort from the patients, care givers and medical staff alike. Furthermore, in some cases, this may cause frustration and lead to pump use withdrawal. Therefore, appropriate assessment and insulin dosing at the initiation of pump therapy leads to earlier improvement in metabolic control, improving long term outcomes, and adherence to treatment.

In clinical practice, most newly diagnosed subjects with type 1 diabetes start with MDI therapy and then switch to pump therapy. All pediatric subjects are potential candidates for pump therapy that can be initiated safely at diagnosis, or at any time thereafter. Pump therapy should be considered in cases of recurrent hypoglycemia, suboptimal diabetes control, wide fluctuations in blood glucose, lifestyle need and diabetes complications, as well as for special populations such as young children, pregnant women, athletes, and subjects who experience the dawn phenomenon [13].

The present invention solves the above-mentioned problem by providing a technique for switching from Multiple Daily Injections to pump therapy and vice versa. The technique may be used by MDI users desiring to utilize advanced closed-loop technology and/or by subjects with type 1 diabetes switching from Multiple Daily Injections to Pump therapy and vice versa. The technique of the present invention may also be useful for healthcare professionals and/or patients to determine insulin dosing for first time pump use and/or MDI use and/or for transition from MDI to pump and vice versa. There is provided an individual diabetes decision support system to determine insulin dosing parameters for initiating new modality of insulin treatment (e.g. MDI or Pump). The technique determines needed insulin parameters for switching therapy from MDI to Pump and vice versa. The technique is also configured for determining the insulin settings and/or basal-bolus dosing when switching from MDI to pump and vice versa respectively. The technique provides an automated determination of insulin dosing parameters based on the previous mode of therapy (MDI or Pump). To this end, a novel insulin dosing program is provided to determine optimal initial insulin dosing parameters switching from one mode of therapy to another. The insulin settings and/or basal-bolus dosing (e.g. insulin basal rates and bolus parameters) are based on collected individual data.

According to a broad aspect of the present invention, there is provided a method for use in disease treatment management. The method comprises the following steps: receiving data indicative of pump treatment parameters; analyzing physiological data during use of a pump; the physiological data being indicative of a physiological characteristic of the patient; analyzing the received pump treatment parameters data to thereby identify at least one patient-related treatment characteristic; creating data indicative of multiple daily injections (MDI) treatment parameters by automatically determining individualized insulin dosing injection parameters data based on the at least one patient-related treatment characteristic and the physiological data.

In some embodiments, the technique comprises analyzing data generated from different devices including: glucose, insulin activity, food etc. in order to determine the needed program of insulin pump therapy out of data collected during MDI therapy and vice versa. Therefore, the physiological data may comprise at least one of: insulin delivery, glucose monitoring data, insulin activity, physical activity, meal event, food type, age, and a metabolic state influencing insulin sensitivity. The metabolic state may comprise at least one of stress, illness, menstrual cycle, hormonal changes and drug consumption. The patient-related treatment characteristic may comprise at least one of insulin response, glucose pattern, meal requirement, personal metabolic profile and insulin requirements including at least one of total daily insulin requirement and differences in insulin requirements during the course of the day. The individualized insulin dosing injection parameters data may comprise at least one of: insulin treatment plan including initial insulin dosing parameters, long acting (basal) insulin dose, number of required doses of basal insulin, time for basal insulin injection, different types of short acting insulin for different times of the day, short acting insulin dose for meals, Carbohydrate ratio (CR) according to the time of day and correction factor (CF) and individual active insulin time (AI) according to the time of day.

In some embodiments, automatically determining long acting (basal) insulin dose comprises analyzing the glucose pattern to define a certain time and a certain number of required doses of the basal insulin. The certain time may be defined as a time of injecting the long acting insulin dose.

In some embodiments, the individualized insulin dosing injection parameters data comprise a short-acting insulin dosage component taken according to a sliding scale.

In some embodiments, the analyzing of the glucose pattern comprises identifying at least one pattern of glucose of different levels, or glucose trend as compared to a patient target glucose level.

In some embodiments, when a plurality of patterns of glucose of different levels or glucose trend is identified, automatically determining long acting (basal) insulin dose comprises splitting the long acting insulin injection accordingly and determining times of injections accordingly.

In some embodiments, the method further comprises analyzing the received pump treatment parameters data and the physiological data to thereby optimize pump treatment parameters data.

In some embodiments, the method further comprises converting the individualized insulin dosing injection parameters data from a specific amount to a sliding scale and vice versa.

In some embodiments, the method further comprises determining an active insulin decay time.

According to another broad aspect of the present invention, there is provided a method for use in disease treatment management. The method comprises receiving data indicative of multiple daily injections (MDI) treatment parameters; analyzing physiological data during multiple daily injections; the physiological data being indicative of a physiological characteristic of the patient; analyzing the received MDI treatment parameters to thereby identify insulin requirements; and creating data indicative of pump treatment parameters by automatically determining individualized insulin dosing pump parameters based on the insulin requirements and the physiological data. The individualized insulin dosing pump parameters may include at least one of: daily insulin basal rate including basal intervals and dose; carbohydrate ratio (CR) according to the time of day; correction factor (CF) according to the time of day; individual active insulin time (AI) according to the time of day; and at least one individual glucose target according to the time of day. The insulin requirements may include at least one of total daily insulin requirement, differences in insulin requirements during the course of the day, glucose patterns, and meal requirements. The data indicative of pump treatment parameters may comprise total daily basal dose and long acting insulin type.

In some embodiments, automatically determining daily insulin basal rate further comprises finding a pattern in patient glucose levels and dividing a day into (N) time periods.

In some embodiments, automatically determining daily insulin basal rate further comprises defining at least one basal pattern being indicative of hourly basal rate at every daily period.

In some embodiments, the method further comprises calculating the carbohydrate ratio, dividing a day into (M) time periods, and changing the carbohydrate ratio proportionally to changes in basal patterns.

In some embodiments, the method further comprises calculating the correction factor and dividing a day into K time periods, and then changing the correction factor proportionally to changes in basal patterns.

In some embodiments, the method further comprises calculating individual active insulin time based on the physiological data and the data indicative of MDI treatment parameters, and dividing a day into L time periods.

In some embodiments, the method further comprises generating individualized insulin dosing pump parameters configured for at least one of the following: for operating a pump, and for presentation on a user interface.

In some embodiments, the MDI treatment parameters are received from at least one of a multiple daily injections (MDI) device, a patient, personal medical data, and a medical practitioner.

In some embodiments, the method further comprises analyzing the received MDI treatment parameters and the physiological data to thereby optimize the received MDI treatment parameters.

Innovative measurements relating to the era of software prescription therapy, enable a personalized individually targeted approach which offers an alternative solution for diabetes management. The technique of the present invention provides automated advisors for insulin dosing titration improving glycemic control, while relieving the burden from patients and healthcare systems. As described above, the technique of the present invention can be of assistance to a professional team during routine follow-up and to patients between visits. The use of the technique of the present invention significantly aids a growing number of patients with diabetes seeking pump therapy. The technique advises on insulin dosing when switching from MDI to pump therapy and vice versa. There is provided a safe, proven technique that individually sets the insulin pump dosing when first starting its use, providing better glycemic control. It optimizes insulin therapy, accelerates the transition process needed for switching to this technology, and increases the adherence to therapy as well as the success of pump therapy. The invention also assists health care providers by saving time, thus relieving a shortage of expert medical professionals. In some embodiments, the technique of the present invention provides a clinical decision support tool for assisting the professional team to accelerate the transition process, save valuable time, and introduce expert-physician-diabetes-decision support to patients around the world. The technique individually tailors the new pump setting, thus decreasing the time needed to achieve stabilization in insulin dosages.

Moreover, there is a need for a personalized approach taking into an account the individual insulin sensitivity and personal glucose-insulin dynamics, which may lead to a transient deterioration in glycemic control. Initiation of pump therapy usually includes educational sessions ranging around 20-40 hours and frequent endocrinologist visits thereafter to adjust insulin doses. On the other hand, established pump users discontinue pump use at a rate of 4-5% mostly due to comfort issues [13, 14]. Others temporarily revert to MDI due to pump malfunction, lifestyle changes (during holidays, camp days, sport and more) and in cases of complications, such as infection. In a recent survey conducted by Pickup J C et al, among pump users, the rate of pump malfunction was high and occurred in 48% of subjects during the first year of use [15]. Similarly, another study found 36% breakdown of the pumps issued to patients [16]. Therefore, subjects who use pump therapy need an alternative regimen of MDI. This may happen at times that no medical guides are available. Moreover, a technique for reversion to MDI for different insulin regimens, accessible to patients at any time, is needed.

This novel technique automatically produces personalized treatment switch recommendation. More specifically, the technique determines a personalized insulin treatment plan including initial insulin dosing settings for switching patients from MDI to pump therapy and vice versa. Up-to-date switching from one treatment modality to another is done manually by a specialized medical practitioner in a non-personalized manner More specifically, the present invention provides a technique for receiving treatment parameters from an insulin pump and transforming the pump treatment parameters to injection treatment parameters and vice versa. The present invention provides a new technique comprising analyzing prior patient data, such as glucose and insulin behavior, and automatically determining personalized initial pump settings (e.g. automatic insulin dose adjustments). Initial pump settings may be based on prior knowledge of the patient's insulin response and/or glucose daily patterns and/or personal metabolic profile. The use of personalized automated initial pump settings improves glycemic control, requiring less subsequent adjustments of pump settings and shortening the duration, to achieve target glucose levels resulting in improved satisfaction and adherence to pump therapy treatment. The technique also determines appropriate dosing when the reverse switch is required, from pump therapy to MDI, minimizing disruption in glycemic control.

The technique of the present invention provides a diabetes decision support system for insulin dosing determination based on retrospective data. More specifically, the technique comprises determining the different optimal insulin dosing parameters (e.g. basal and bolus) needed for switching from one mode of therapy to the other (MDI vs. Pump and vice versa) based on retrospective data.

According to another broad aspect of the present invention, there is provided a control unit for use in disease treatment management. The control unit comprises a data processor utility configured and operable as an advisor utility for carrying out the method as described above.

According to another broad aspect of the present invention, there is provided a system for use in disease treatment management. The system comprises a control unit configured for receiving physiological data being indicative of a physiological characteristic of a patient and one of the following: data indicative of pump treatment parameters or data indicative of multiple daily injections (MDI) treatment parameters; wherein in a first operative mode the processing utility is configured for processing the data indicative of the pump treatment parameters to thereby create data indicative of MDI injection treatment parameters, and, in a second operative mode, the processing utility is configured for processing the data indicative of the MDI treatment parameters to thereby create data indicative of pump treatment parameters. The processing utility may be configured and operable for identifying at least one of insulin response, glucose pattern, meal requirement, personal metabolic profile and insulin requirements including at least one of total daily insulin requirement and differences in insulin requirements during the course of the day.

In some embodiments, the system comprises a control unit having (1) a data input utility configured and operable to receive data from various sources such as self-monitored blood glucose (SMBG) or continuous glucose monitor (CGM) or pump or MDI and (2) a data processor utility configured for analyzing data generated from the different devices including: glucose, insulin, activity, food etc. in order to determine a needed program of insulin pump therapy out of data collected during MDI therapy and vice versa. The data input utility may thus be connected (wired or wireless) to the output data of SMBG or CGM for glucose monitoring data and insulin delivery data collected from pump or pens (during treatment or not) and may include other inputs collected from connected sensors and other sources data (e.g. food, user characteristics such as insulin sensitivity retrospective data) collected during MDI or pump therapy to be used in order to switch from one mode of therapy to the other.

In some embodiments, the system may comprise a data output utility configured and operable to provide recommendation data regarding the setting of the insulin pump or of the multiple daily injections device. It may provide insulin pump settings including insulin dosing parameters, initial insulin parameters, carbohydrate ratio (CR), correction factor (CF), basal rate and insulin activity time for patients switching to pump therapy. For patients switching to MDI therapy, the data output utility may be configured and operable to provide initial insulin parameters including basal insulin and bolus dosing.

The term “pump treatment parameters” refers hereinafter to data received directly or indirectly from a pump device including daily basal dose and bolus calculator parameters such as carbohydrate ratio, correction factor, glucose target and insulin activity time.

In some embodiments, when the data input utility receives data indicative of multiple daily injections (MDI) treatment parameters, the recommendation data may comprise at least one of insulin dosing pump parameters including at least one of: daily insulin basal rate including basal intervals and dose; at least one carbohydrate ratio (CR) and correction factor (CF) according to the time of day; at least one individual insulin activity time (AI) according to the time of day, and at least one glucose target value according to the time of day; wherein calculation of the recommendation data is based on the data indicative of multiple daily injections (MDI) treatment parameters.

In other embodiments, when the data input utility receives data indicative of pump treatment parameters, the recommendation data may comprise at least one of insulin treatment plan including initial insulin dosing parameters, long acting (basal) insulin dose, number of required doses of basal insulin, time for basal insulin injection, different types of short acting insulin for different times of the day, short acting insulin dose for meals and correction including Carbohydrate ratio (CR) and correction factor (CF) according to the time of day and at least one glucose target value according to the time of day.

In some embodiments, the system further comprises a memory utility for storing the physiological data and/or the data indicative of pump treatment parameters obtained over a certain time and/or the data indicative of MDI treatment parameters obtained over a certain time.

In some embodiments, the control unit is configured and operable for converting the individualized insulin dosing injection parameters data from a specific amount to a sliding scale and vice versa.

In some embodiments, the control unit is configured and operable for determining an active insulin decay time.

In some embodiments, the control unit is configured and operable for determining the long acting (basal) insulin dose by analyzing a glucose pattern to define a certain time and a certain number of required doses of the basal insulin.

As described above, according to a broad aspect of the present invention, there is provided a computer program recordable on a storage medium and comprising a machine readable format. The computer program is configured and operable, when being accessed, to carry out the following: receiving and processing physiological data being indicative of a physiological characteristic of the patient and data indicative of multiple daily injections (MDI) treatment parameters or data indicative of pump treatment parameters to thereby create in a first operative mode data indicative of MDI injection treatment parameters for MDI users, and, in a second operative mode, data indicative of pump treatment parameters for pump users.

According to a broad aspect of the present invention, there is provided a computer program product, comprising a non-transitory tangible computer readable medium having computer readable program code embodied therein. The computer readable program code is adapted to be executed to implement a method as described above.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A is a schematic block diagram illustrating the system of the present invention;

FIG. 1B is an example of a schematic block diagram illustrating the system according to some embodiments of the present invention;

FIG. 2A-2B show schematic possible configurations of the system of the present invention for transition from MDI to pump therapy (FIG. 2A) and from pump to MDI therapy (FIG. 2B);

FIG. 3 is a graph illustrating the average basal rate and the basal pattern changed with respect to the glucose pattern by using the teachings of the present invention; and

FIG. 4 is a graph illustrating insulin glargine active profile in a patient with T1DM.

### DETAILED DESCRIPTION OF EMBODIMENTS

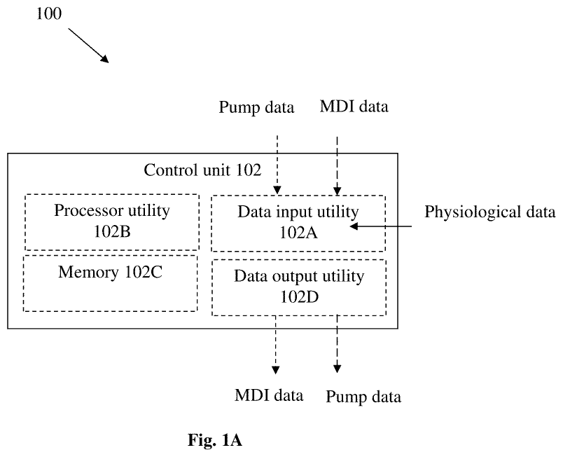

Reference is made to FIG. 1A exemplifying a schematic block diagram of the system 100 of the present invention. System 100 is configured for use in disease treatment management. System 100 comprises a control unit 102 configured for receiving data indicative of a physiological characteristic of a patient. The physiological data may comprise at least one of: insulin delivery, glucose monitoring data, physical activity, meal event, food type, age, and a metabolic state influencing insulin sensitivity. Each of the above physiological parameters can contribute to configuration of the pump or MDI treatment.

In a specific and non-limiting example:
1. The physical activity may help to determinate if the changes in the sensitivity that followed by glucose changes should be reflected in the patient treatment parameter (for example, by modifying the correction factor).
2. The age and metabolic state may contribute to anticipate the insulin requirement of a certain patient before calculating the different sensitivity parameters based on the insulin delivery and/or glucose monitoring data.

In a first operative mode, control unit 102 receives data indicative of pump treatment parameters and processes the pump data to thereby create data indicative of MDI injection treatment parameters (for MDI users). In a second operative mode, control unit 102 receives data indicative of MDI treatment parameters and processes the MDI data to thereby create data indicative of pump treatment parameters (for pump users). More specifically, control unit 102 comprises a data input utility 102A configured and operable to receive physiological data being indicative of a physiological characteristic of a patient and data indicative of pump treatment parameters or data indicative of multiple daily injections (MDI) treatment parameters, a processor utility 102B being configured and operable to process the data, and a data output utility 102D being configured and operable to provide recommendation data regarding the setting of the insulin pump or of the multiple daily injections device. The recommendation data may include the setting of the insulin pump or of the multiple daily injections device but also behavioral recommendations (e.g. to give a bolus for every meal and snack). The input data may be stored in any external device such as a notebook, any available web application (a client server computer program), or a manual report. The control unit 102 may comprise a memory utility 102C being configured and operable to store the physiological data and/or data indicative of pump treatment parameters obtained over a certain time and/or data indicative of multiple daily injections (MDI) treatment parameters obtained over a certain time and/or the recommendation data.

In the first operative mode, the recommendation data is individualized insulin dosing injection parameters data based on at least one patient-related treatment characteristic and the physiological data. The individualized insulin dosing injection parameters data comprises an insulin treatment plan including initial insulin dosing parameters and/or long acting (basal) insulin dose and/or number of required doses of basal insulin and/or time for basal insulin injection and/or different types of short acting insulin for different times of the day and/or short acting insulin dose for meals and correction in the form of Carbohydrate ratio (CR) and correction factor (CF) or in a sliding scale according to the time of day and/or glucose target value according to the time of day.

In the second operative mode, the recommendation data comprises at least one of insulin dosing pump parameters including daily insulin basal rate including basal intervals and dose and/or at least one carbohydrate ratio (CR) and correction factor (CF) according to the time of day and/or at least one individual insulin activity time (AI) according to the time of day and at least one glucose target value according to the time of day. The calculation of the recommendation data is based on the data indicative of multiple daily injections (MDI) treatment parameters.

In some embodiments, control unit 102 is configured and operable for identifying at least one of insulin response, glucose pattern, meal requirement, personal metabolic profile and insulin requirements including at least one of total daily insulin requirement and differences in insulin requirements during the course of the day.

The control unit 102 may comprise a transceiver permitting to be connected to a communication unit and to transmit and/or receive data. In some embodiments, the control unit 102 may be configured as an electronic module for collecting and processing data. It should be noted that all required operations may be controlled by means of a processing utility, such as a DSP, microcontroller, FPGA, ASIC, etc., or any other conventional and/or dedicated computing unit/system. The term “processing utility” should be expansively construed to cover any kind of electronic device with data processing capabilities, including, by way of non-limiting example, personal computers, servers, computing systems, communication devices, processors (e.g. digital signal processor (DSP), microcontrollers, field programmable gate array (FPGA), application specific integrated circuit (ASIC), etc.) and other electronic computing devices. The processing utility may comprise a general-purpose computer processor, which is programmed in software to carry out the functions described hereinbelow. Also, operations in accordance with the teachings herein may be performed by a computer specially constructed for the desired purposes or by a general purpose computer specially configured for the desired purpose by a computer program stored in a computer readable storage medium. The control unit includes inter alia a signal generator and at least one utility part (suitable software and/or hardware) for generating a signal indicative of recommendation data regarding the setting of the insulin pump or of the multiple daily injections device. The different elements of the control unit (electronic unit and/or mechanical unit) are connected to each other by wires, or are wireless. The software may be downloaded to the processing utility in electronic form, over a network, for example, or it may alternatively be provided on tangible media, such as optical, magnetic, or electronic memory media. Alternatively or additionally, some or all of the functions of the control unit may be implemented in dedicated hardware, such as a custom or semi-custom integrated circuit or a programmable digital signal processor (DSP).

In some embodiments, there is provided a computer program recordable on a storage medium and comprising a machine readable format. The computer program product may comprise a non-transitory tangible computer readable medium having computer readable program code embodied therein, the computer readable program code adapted to be executed to implement a method as described below. The computer program is configured and operable, when being accessed, to carry out the following: receiving and processing physiological data being indicative of a physiological characteristic of the patient and data indicative of multiple daily injections (MDI) treatment parameters or data indicative of pump treatment parameters, to thereby create in a first operative mode data indicative of MDI injection treatment parameters for MDI users, and in a second operative mode, data indicative of pump treatment parameters for pump users.

The technique of the present invention may use information from an existing third party diabetes management system such as a secured and HIPAA (Health Insurance Portability and Accountability Act)-compliant diabetes data management platform. The data input includes at least one of insulin delivery, glucose levels, food, physical activity and any information available from a patient's devices. Following data collection (e.g. downloaded from the personal devices), the gathered information is analyzed by the processing utility 102 to identify at least one of insulin requirements (total daily requirements and differences during the course of the day), glucose patterns, glucose trends, meal insulin requirements, insulin treatment patterns, carbohydrates consumption etc. The efficacy of the patient's glucose control may be verified according to the known glucose goals recommended by the ADA [17] or set individually. The data output may be displayed to the patient as a form of a report with recommendations defining how to set the individual insulin pump settings or how to set the MDI regimen. The recommendations may be given directly to the patients and other care givers via an application and/or a web site. The recommendations may be sent to a cloud by the transceiver, to allow remote counsel capabilities.

Reference is made to FIG. 1B exemplifying a schematic block diagram of a possible configuration of the system of the present invention. The system 200 is implemented by a software product configured for assisting in insulin dosage decision making. The control unit 102 of FIG. 1A may comprise at least one of the following two components: a pump to MDI switch advisor 202 (referred in the figure as switch P2M) for patients who use insulin pump therapy and desire to switch to MDI and an MDI to pump switch advisor 204 (referred in the figure as switch M2P) for patients who use MDI therapy and desire to switch to insulin pump. This system is intended as a tool for physicians and/or for patients and/or caregivers.

The pump to MDI switch advisor 202 is configured to transform any given pump settings into an MDI treatment regimen. The pump settings may include the basal rate plan and bolus calculator plan. The bolus calculator plan may include the carbohydrate ratio, correction factor, active insulin time and glucose target. The MDI treatment regimen may include a plan for the long acting insulin (type of insulin and time (or times) of injections) and a plan for the short acting insulin that includes either a CR/CF or sliding scale plan.

More specifically, the input data of the pump to MDI switch advisor 202 comprises at least one of (i) pump settings including correction factor (CF), carbohydrates ratio (CR), glucose target, active insulin time and/or basal rate, (ii) glucose data including at least one of continuous glucose monitor (CGM), flash glucose monitoring (FGM) and self-monitored blood glucose (SMBG), (iii) insulin data including at least one of basal records and bolus records, (iv) carbohydrates data and (v) personally determined type of basal and boluses insulin.

The output data comprising the pump to MDI switch advisor 202 comprises at least one of (i) long acting plan including timing amount and type (ii) short acting plan including a treatment mean in the form of CR/CF or in a sliding scale and type as described further below. In this context, the type of the treatment refers to regular insulin or insulin analogs depending on the typical meals taken by the patient.

The MDI advisor 204 is configured to transform any given MDI treatment plan into settings to be programmed inside an insulin pump. The MDI treatment regimen may include a plan for the long acting insulin (type of insulin and time/s of injections) and a plan for the short acting insulin that includes either a CR/CF or sliding scale plan. The pump settings, as output of the MDI advisor 204, may include the basal rate plan and bolus calculator plan. The basal rate plan may include an hourly basal dose for the whole day. The bolus calculator plan may include the carbohydrate ratio, correction factor, active insulin time and glucose target.

More specifically, the input data of the MDI advisor 204 comprises at least one of (i) long acting plan including timing and amount, number and type (ii) short acting plan including a treatment mean in the form of CR/CF or in a sliding scale as described further below and type, (iii) glucose data including at least one of continuous glucose monitor (CGM), flash glucose monitoring (FGM) and self-monitored blood glucose (SMBG), (iv) insulin data including at least one of basal records and bolus records and (v) carbohydrates data.

The output data comprising MDI advisor 204 comprises pump settings including at least one of correction factor (CF), carbohydrates ratio (CR), glucose target, active insulin time and basal rate.

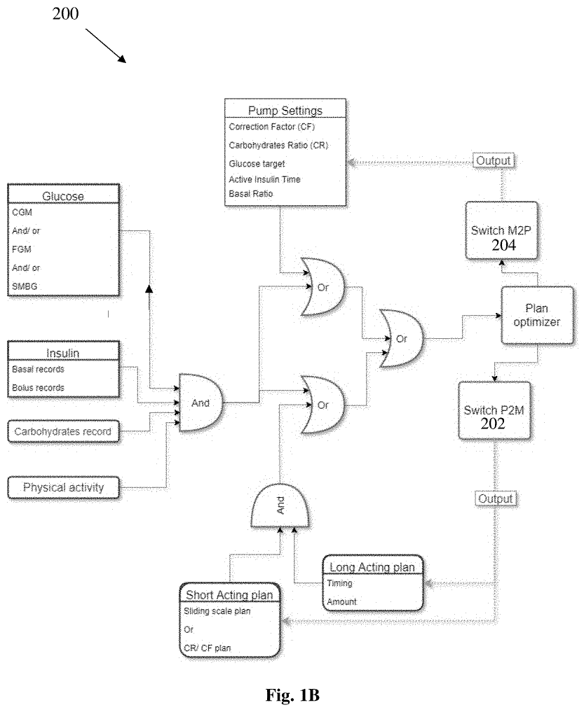

Reference is made to FIGS. 2A-2B exemplifying some possible methods for use in disease treatment management according to some embodiments of the present invention. In this specific and non-limiting example shown in FIG. 2A, illustrating the second operative mode, physiological data such as food ingested (e.g. time, amount, content) and/or physical activity (e.g. time, duration, intensity) during MDI injection treatment, blood glucose monitoring and insulin treatment/delivery (e.g. regimen, time, amount) are received by the data input utility 102A of FIG. 1 being illustrated here as a diabetes management system(s). Although these specific types of physiological data are illustrated, the invention is not limited to this specific data and also may include meal event, food type, age, and a metabolic state influencing insulin sensitivity. The metabolic state may comprise at least one of stress, illness, menstrual cycle, hormonal changes and drugs consumption. The control unit processes the received data (algorithm analysis), and analyzes the received pump treatment parameters data to thereby identify at least one patient-related treatment characteristic. The control unit creates data indicative of multiple daily injections (MDI) treatment parameters by automatically determining individualized insulin dosing injection parameters data based on the at least one patient-related treatment characteristic and the physiological data, and provides personalized insulin pump settings including basal plan, carbohydrate ratio (CR), correction factor (CF) and insulin activity time. In the figure, the personalized insulin pump settings illustrated are activity time, carbohydrate ratio, basal plan and correction factor, however, the individualized insulin pump settings are not limited to such parameters they can also include the way the bolus wave is taken (normal, square or dual).

In a specific and non-limiting example shown in FIG. 2B, illustrating the first operative mode, physiological data such as food ingested (e.g. time, amount, content) and/or physical activity (e.g. time, duration, intensity) during use of a pump, blood glucose monitoring and insulin pump treatment (e.g. including basal plan, carbohydrate ratio (CR), correction factor (CF) and insulin activity time) are received by the data input utility 102A of FIG. 1 being illustrated here as a diabetes management system(s). Although these specific types of physiological data are illustrated, the invention is not limited to this specific data and also may include meal event, food type, age, and a metabolic state influencing insulin sensitivity. The control unit processes the received data and suggests a personalized MDI regimen including at least one of: basal insulin (amount and number of required doses of basal insulin, the ideal time for basal insulin injection) and meal time insulin. The recommendations may be accompanied by an explanation for the suggested treatment plan. For example, the technique may be used as a tool for physicians. Therefore the physician can approve, reject or change the recommendations and issue an update treatment plan to the patient.

### EXAMPLES

Various examples were carried out to prove the embodiments claimed in the present invention. Some of these experiments are referred to hereinafter. The examples describe the manner and process of the present invention for carrying out the invention, but are not to be construed as limiting the invention.

In the first operative mode (from pump to MDI), the data input utility may receive the following pump settings for a specific patient illustrated in Tables 1-5 below:

TABLE 1
Correction Factor

| Time | Ratio |
|---|---|
| (HH:MM-HH:MM) | (Units/mg/dL) |
| 00:00-06:00 | 80 |
| 06:00-11:00 | 50 |
| 11:00-18:00 | 40 |
| 18:00-24:00 | 45 |

TABLE 2
Carbohydrate Ratio

| Time | Ratio |
|---|---|
| (HH:MM-HH:MM) | (grams/Units) |
| 00:00-07:00 | 20 |
| 07:00-13:00 | 10 |
| 13:00-17:00 | 15 |
| 17:00-24:00 | 12 |

TABLE 3
Glucose target

| Time | Low-High |
|---|---|
| (HH:MM-HH:MM) | (mg/dL) |
| 00:00-24:00 | 110-130 |

TABLE 4
Active insulin time

| Time | |
|---|---|
| (HH:MM-HH:MM) | Hours |
| 00:00-24:00 | 2 |

TABLE 5
Basal - Plan

| Time | Rate |
|---|---|
| (HH:MM-HH:MM) | (Units/Hours) |
| 00:00-03:00 | 1 |
| 03:00-07:00 | 1.3 |
| 07:00-10:00 | 1 |
| 10:00-15:00 | 1.2 |
| 15:00-18:00 | 0.9 |
| 18:00-21:00 | 1.3 |
| 21:00-24:00 | 1.1 |

Table 1 shows the correction factor plan that includes the time and correction factor in units/mg/dl as programmed inside the pump. Table 2 shows the carbohydrates ratio that includes the time and the ratio in grams/units as programmed inside the pump. Table 3 shows the glucose target range that includes the time and glucose low and high target as programmed inside the pump. Alternatively, the glucose target plan may include correction threshold and glucose target instead of the low and high glucose targets. Table 4 shows the active insulin time in hours as programmed inside the pump. Table 5 shows the basal plan that includes the time and rate in units/hours as programmed inside the pump. The data input utility may also receive data such as: glucose readings, insulin records, carbohydrate intake, physical activity, illness etc.

The data output utility provides a treatment plan for MDI regimen. It can include, in a specific and non-limiting example, as illustrated in Tables 6-10 below:

TABLE 6
Correction Factor

| Time | Ratio |
|---|---|
| (HH:MM-HH:MM) | (Units/mg/dL) |
| Morning | 60 |
| 07:00-12:00 | 45 |
| 12:00-18:00 | 35 |
| 18:00-21:00 | 40 |
| 21:00-24:00 | 45 |

TABLE 7
Carbohydrate Ratio

| Time | Ratio |
|---|---|
| (HH:MM-HH:MM) | (grams/Units) |
| 00:00-07:00 | 18 |
| 07:00-13:00 | 8 |
| 13:00-16:00 | 15 |
| 17:00-21:00 | 13 |
| 21:00-24:00 | 15 |

TABLE 8
Glucose target

| Time | Low-High |
|---|---|
| (HH:MM-HH:MM) | (mg/dL) |
| 00:00-24:00 | 100-130 |

TABLE 9
Active insulin time

| Time | |
|---|---|
| (HH:MM-HH:MM) | Hours |
| 00:00-24:00 | 3 |

TABLE 10
Long acting insulin

| Injection Time | Amount |
|---|---|
| Morning (or 8AM) | 30 Units |

As shown in Table 6, the amount (in units/mg/dl) and the time interval of the suggested correction factor is provided. As shown in Table 7, the amount (in grams/units) and the time interval of the suggested carbohydrate ratio is provided. As shown in Table 8, the suggested glucose target range for 24 hours is provided. As shown in Table 9, the suggested active insulin time for 24 hours is provided. As shown in Table 10, the amount and the time of the suggested long acting insulin is provided.

Alternatively, the data output can be provided in terms of a sliding scale, as illustrated below in Table 11 and Table 12:

TABLE 11
Bolus plan in sliding scale

| Day time | Plan | |
|---|---|---|
| Morning | 4 Units | When BG < 150 |
| | 5 Units | When 150 ≤ BG ≤ 250 |
| | 6 Units | When 250 ≤ BG ≤ 350 |
| Noon | 5 Units | When BG < 150 |
| | 6 Units | When 150 ≤ BG ≤ 250 |
| | 7 Units | When 250 ≤ BG ≤ 350 |
| Evening | 4 Units | When BG < 150 |
| | 5 Units | When 150 ≤ BG ≤ 250 |
| | 6 Units | When 250 ≤ BG ≤ 350 |
| Night | 3 Units | When BG < 150 |
| | 4 Units | When 150 ≤ BG ≤ 250 |
| | 5 Units | When 250 ≤ BG ≤ 350 |

TABLE 12
Long acting insulin

| Injection Time | Amount |
|---|---|
| Morning (or 8AM) | 30 Units |

The same example can be given for the second operative mode; where the input data comprises the treatment plan for the MDI regimen (either in CR/CF terms or sliding scale) or/and data such as patient-related treatment characteristic glucose readings, insulin records, carbohydrate intake, physical activity, illness etc. The output data comprises the pump settings that include the CR, CF, target, active insulin time and basal plans.

As described above, the treatment plan according which the amount of bolus of insulin given for carbohydrates intake or correction of high blood glucose (BG) is determined may be provided as a CR/CF plan or as a sliding scale. In this connection, it should be noted that the use of the CR/CF calculation is more common among pump users. This is because the pump includes a bolus calculator feature that helps to automatically calculate the amount of insulin that needs to be delivered, based on the CR/CF/AI/Target values. In addition, usually these patients know how to estimate the amount of carbohydrates in a meal. This is in contrast to an MDI patient who is less likely to be trained on carbohydrate counting. Patients who change from pump to MDI treatment may continue using this CR/CF method while blousing using a syringe (i.e. the patient will keep using a bolus calculator that takes into account the CR/CF for calculating the amount of insulin). The use of the sliding scale is more common among MDI patients. This is due to the lack of ability to make accurate calculations based on carbohydrate amount, CR/CF and other contributing factors.

In some embodiments, the control unit of the present invention is configured and operable for converting the treatment plan from a CR/CF plan to a sliding scale and vice versa.

The amount of insulin that should be given can be calculated as follows:

$$Bolus = \frac{Carbohydrates}{CR} + \frac{BG - Target}{CF} - {AI}^{*}$$

when using a pump, the Active Insulin (AI) being the insulin assumed to be active from the previews boluses, which might be reduced from the amount of given insulin.

For example, in case a patient who eats 30 grams of carbohydrates and has glucose levels of 200 mg/dL with the following factors: CR=10 [gr/U], CF=50 [mg/dL/U], Target=[100 mg/dL]. This amounts to a bolus of 5 units of insulin, 3 units for the carbohydrates intake and 2 additional units for correcting the high glucose levels.

When the patient is using a more heuristic matric for determining the amount of insulin needed to be delivered, such as a sliding scale approach, the amount of insulin is determined according to the blood glucose value at the time of the bolus. It may include a fixed dose and additional increment depending on the glucose value.

For example, typically, in case the patient eats, and the glucose level is 200 mg/dL with the following sliding scale:
If glucose level<150, give 3 Units of insulin
If glucose level is between 151 and 250, give 4 Units of insulin
If glucose level is between 251 and 350, give 5 Units of insulin

As follows from the sliding scale above, the patient will deliver a bolus of 4 units for the given glucose level at the time of the bolus. It should be noted that in contrast to the previous example, in this example the patient usually does not count the amount of carbohydrates in the meal.

By using the teachings of the present invention, the pump to MDI switch advisor may convert the CR/CF plan into sliding scale as follows:

In case the only input data is the pump settings (CR/CF plan), with no glucose, insulin or carbohydrate intake data, the plan conversion may be carried out as follows:

First, the CR is converted to a fixed dose by estimating the common carbohydrate intake. This can be done by either using a constant amount driven from general population carbohydrate intake, or by getting this information from the patient/physician:

$${Fixed}_{Dose} = \frac{common Carbs}{CR}$$

Then, the increment value (U_{Increment}) is defined with respect to the patient CF value accordingly:

$${U}_{Increment} = max \{ {Round}_{x} [ Ave ( \frac{( {Glucose}_{threshold} + {Range}_{Inc} ) - Target}{CF} , \frac{( {Glucose}_{threshold} - Target}{CF} ) ] , {Round}_{x} [ \frac{( {Glucose}_{threshold} - {low}_{Target}}{CF} ] \}$$

where, Roundₓ is an operator that rounds the value to the nearest x value, Glucose_{threshold} is the glucose value from which the patient should start adding insulin to correct the glucose levels (e.g. default range can be

$$50 \frac{mg}{dL} ) ,$$

Range_{Inc} defines the steps in the glucose levels (e.g. default value can be

$$150 \frac{mg}{dL} ) ,$$

Target is the glucose target (e.g. default value can be 100 mg/dL), low_{target} is the low glucose level to ensure that the increment value will not result in a risk of low glucose value (e.g. default value for the low_{target} can be 70 mg/dL).

The final sliding scale may be defined as follows:

$$Bolus = \{ \begin{matrix}Fix\_Dose & {Glucose}_{threshold} > BG \geq {Glucose}_{threshold} + {Range}_{inc} , where N = 0 \\ {Fix}_{Dose} + ( N - 1 ) \times {U}_{increment} & \begin{matrix}{Glucose}_{threshold} + N \times {Range}_{inc} > BG \geq {Glucose}_{threshold} + {Range}_{inc} + \\ N \times {Range}_{inc} , where N ∈ [ 1 … M ]\end{matrix}\end{matrix}$$

For example, for the following pump settings as defined below in Table 13 and a common carbohydrate amount of 40 grams.

TABLE 13

| | | |
|---|---|---|
| CR | 10 | [grams/U] |
| CF | 25 | [mg/dL/U] |
| Low target-High target | 90-130 | mg/dL |
| AI | 2 | hours |

The converted resulted sliding scale may be:

$$Bolus = \{ \begin{matrix}4 & when BG \leq 150 \\ 7 & When 150 < BG \leq 200 \\ 10 & when 200 < BG \leq 250 \\ ⋮ & ⋮\end{matrix}$$

The sliding scale might be changed throughout the day in case the CR/CF plan includes multiple daily ratios.

In case the input data is the glucose, insulin and carbohydrates data, in addition to the pump settings, the plan conversion may be carried out as follows: determining the Fix_Dose in a similar way as above, where the common carbohydrates intake is calculated from the actual patient carbohydrates records; determining the initial U_{Increment} in a similar way as above, adjusting both Fixed_Dose and U_{Increment} by calculating the insulin dose based on the Fixed_Dose and U_{Increment} for every bolus in the history; estimating the bolus amount (i.e. what should have been the ideal amount of insulin that would bring the glucose level closer to the target value) for every bolus in the history, calculating the ratio of insulin delivered to correct the glucose levels versus the insulin delivered to treat the carbohydrate intake, for every bolus, adjusting the Fixed_Dose and U_{Increment} according to the difference between the calculated insulin dose, the estimated bolus and the ratio of insulin delivered. It should be noted that by knowing the carbohydrates intake at each time, the control unit can divide the day into sections according to the carbohydrate values. These sections can be used for creating several sliding scale plans throughout the day.

By using the teachings of the present invention, the MDI to pump switch advisor may convert the sliding scale into a CR/CF plan as follows:

In case the only input data is the sliding scale plan, with no glucose, insulin or carbohydrate intake data, the plan conversion may be carried out as follows: first, the Fixed_Dose part in the sliding scale is converted to the CR dose by estimating the common carbohydrate intake. This can be done by either using a constant amount driven from general population carbohydrate intake, or by getting this information from the patient/physician as defined in the following formula:

$$CR = \frac{common Carbs}{Fixed\_Dose}$$

Then, the increment CF value is defined according to the increment value (U_{Increment}) accordingly:

$$CF = max \{ {Round}_{x} [ Ave ( \frac{( {Glucose}_{threshold} + {Range}_{inc} ) - Target}{{U}_{increment}} , \frac{( {Glucose}_{threshold} - Target}{{U}_{increment}} ) ] , \frac{{Glucose}_{threshold} - {low}_{Target}}{{U}_{increment}} \}$$

where, Roundₓ is an operator that rounds the value to the nearest x value, Glucose_{threshold} is the glucose value from which the patient should start adding insulin to correct the glucose levels, Range_{Inc} is the step in the glucose levels for increasing the insulin dose, Target is the glucose target (e.g. default value can be 100 mg/dL), low_{target} is the low glucose level to ensure that the increment value will not result in a risk of low glucose values (e.g. default value for the low_{target} can be 70 mg/dL).

It should be noted that the CR/CF ratios might be changed throughout the day in case the sliding scale plan includes multiple daily changes.

In case the input data is the glucose, insulin and carbohydrates data, in addition to the sliding scale, the plan conversion may be carried out as follows: determining the CR in a similar way as determined above, where the common carbohydrates intake is calculated from the patient's carbohydrates records (if applicable), determining the initial CF in a similar way as determined above, adjusting both CR and CF by calculating the insulin dose based on the CR and CF for every bolus in the history, estimating the bolus amount (i.e. what should have been the ideal amount of insulin that would bring the glucose level to better range) for every bolus in the history, calculating the ratio of insulin delivered to correct the glucose levels versus the insulin delivered to treat the carbohydrate intake for every bolus, adjusting the CR and CF according to the difference between the calculated insulin dose and the estimated bolus amount, and the ratio of delivered insulin.

In some embodiments, the control unit is configured and operable to determine the optimal long acting insulin injection time during the day. The input data may include the history of daily insulin dose (i.e. basal amount delivered by pump or syringe) and the history of glucose levels (either CGM, FGM or SMBG). The history of daily insulin dose enables to calculate the average hourly basal rate throughout the day. For example, injection of 24 units of long acting insulin can be transformed into a basal rate of 1 unit per hour. Or, in case of the basal data being provided via a pump, the hourly basal rate is the average of the hourly basal rates. Then, for modifying the recommendation of the daily insulin basal dose, the control unit may calculate the estimated needed total daily basal dose by using the current patient Total Daily Dose (TDD) as follows:

totalDailyBasal=mTDD×LongActing2TDDRatio×I

where mTDD is the modified patient total daily dose, LongActing2TDDRatio is the ratio between the long acting insulin to the TDD (for example, delivering 10 U of long acting insulin a day with 20 U of rapid acting insulin will provide a ratio of 1/3) and I is a ratio which depends on the long acting insulin type, Iϵ[0.7, 1].

The modified total daily dose may be calculated as:

$$mTDD = TDD + \frac{Ave . BG - Goal . BG}{SafeSensitivity}$$

where TDD is the patient total daily dose, Ave. BG is the patient average glucose levels, Goal. BG is the target glucose levels as defined by the ADA recommendation [18] and SafeSensitivity is the less aggressive sensitivity that is calculated as follows:

$$SafeSensitivity = \frac{2100}{T D D}$$

The control unit may then estimate what should be the required change(s) of the hourly basal rates (modified hourly basal rates) by knowing the history of glucose levels and insulin delivery. In a general manner, it is the determination that when high glucose levels are observed, the hourly basal rates in a shifted time should rise, and vice versa for low glucose levels. The shifted time reflects the time delay of the insulin on the patient glucose levels which may vary between patients. This process changes the hourly basal insulin pattern.

The maximal and minimal hourly basal rates may be defined as the limits of the method as follows:

$$\begin{matrix}maxHourlyBasal = \frac{totalDailyBasal}{24} \times ( 1 + highLimit ) , highLimit \\ ∈ [ 0 , 0.4 ] minHourlyBasal \\ = \frac{totalDailyBasal}{24} \times ( 1 - lowLim ) , lowLimit ∈ [ 0 , 0.4 ]\end{matrix}$$

$$minHourlyBasal = \frac{totalDailyBasal}{24} \times ( 1 - lowLim ) , lowLimit ∈ [ 0 , 0.4 ]$$

Then, for finding the optimized secretion of basal rates, the method may comprise the following steps:

i. Finding a pattern in the patient glucose levels and dividing the day into (N)periods using the k-Means technique. Hours with similar glucose patterns will count as periods in the day.

ii. Defining the hourly basal rate at every daily period as follows:

$${PeriodicBasal}_{i , i ∈ [ 1 , N ]} = min \{ max [ \frac{totalDailyBasal - {\sum }_{n = 1}^{i} {PeriodicBasal}_{n}}{{PeriodHours}_{i}} \times \frac{Ave . {Glucose}_{periodic}}{Ave . {Glucose}_{total}} , minHourlyBasal ] , maxHourlyBasal \}$$

In some embodiments, the method provides a recommendation of the patient's CR. To this end, the method may use the patient's mTDD as described above, and use the modified ‘450’ rule for calculating the primary CR(pCR):

$$pCR = \frac{450 \times \frac{1 - LongActing2TDDRatio}{LongActing2TDDRatio}}{mTDD \times ( 1 - LongActing2TDDRatio )}$$

This pCR may then be adjusted according to the glucose records as well as the carbohydrate records as follows: each meal event is checked for its validity depending on at least one of the following: availability of the glucose levels at the time of the meal and at a time period in the range of between about 2.5-4.5 hours post meal; the emergence of interference factors such as a hypo event before the meal, second bolus injection in between the start and end glucose records, detection of a late bolus event etc. These valid events determine the insulin sensitivity for the meal event according to the post meal effect on the glucose levels. The processing utility decides accordingly if there is a need to increase or decrease the pCR at different times of the day that depend on the times of the meals (breakfast, lunch and dinner in general). It should be noted that the system may use the primary CR as the recommended CR in case this record is not available.

In some embodiments, the method provides recommendation of the patient's CF. To this end, the system may use the recommended CR for recommending also on the patient's primary CF. For doing so, it may use the ‘3’ rule:

pCFᵢ=CRᵢ×3

where i is the CF corresponding to the relevant period of the day as defined by the carbohydrate ratio calculator.

This pCFᵢ may then be adjusted according to the correction boluses that were found in the patient records i.e. the boluses with high glucose level and no carbohydrates record.

The system may use these boluses and the resulting glucose levels at a time period after the injection time to estimate if pCF should be decreased or increased.

In some embodiments, the method provides individual active insulin time (AI) varying across different times of day.

The patient's active insulin time (in hours) may be defined according to the patient's age:

$$AI = \{ \begin{matrix}3 , ∈ [ ( Age < 6 ) OR ( Age > 18 AND \frac{TDD}{Weight} \leq 1 ) ] \\ 2 , ∈ [ ( 6 < Age < 18 ) OR ( \frac{TDD}{Weight} > 1 ) ]\end{matrix}$$

For obtaining pump settings optimization, the method may comprise adjusting the current patient settings (CR, CF, AI and basal rates) using the dedicated technique that is configured for adjusting the treatment for pump users. After adjusting the patient pump settings, the method may use this information for recommending the long acting insulin amount as follows:

LongU=I×basalRatio×mTDD

Where I is the ratio from the rapid acting insulin that is being used in the pump to the long acting insulin that will be used (∈[0.7,1.3]), the basalRatio is the ratio of the patient basal amount out of the patient TDD and mTDD is the patient modified TDD that was calculated in the same manner as was described above.

Then, after calculating LongU, the system may use a patient-related treatment characteristic such as the glucose profile to define what is the best time and the best number of required doses of the basal insulin. It may do so by finding patterns of high glucose or glucose increments as related to the patient average glucose levels at times of non-meaningful interruptions that may be caused mainly by a bolus injection or untreated meals. These can be referred to as the ‘clean’ glucose levels. If such time is found, the system may recommend this time as the best time for injecting the long acting insulin dose. If there is more than one significant high/increment glucose pattern, it may recommend splitting the long acting insulin injection according to these times.

In some embodiments, the method provides short acting insulin doses for meals and correction: such as CR, CF or other sliding scale treatments. Different types of short acting insulin for different times of the day etc., may be provided as deemed necessary for the individual patient.

In addition to treatment switch, the system can also perform treatment optimization. i.e. treatment parameters are optimized to improve patient glycemic state, and then switch to a different treatment modality.

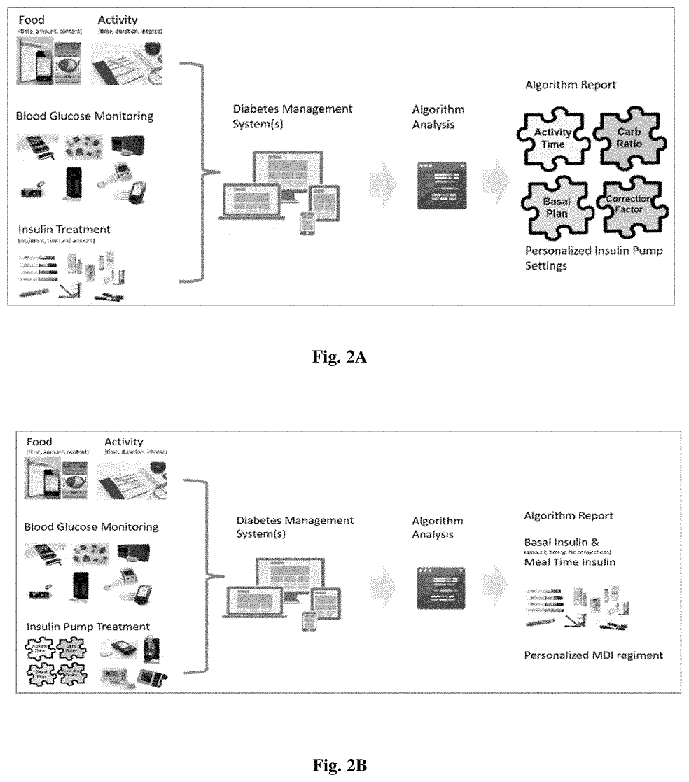

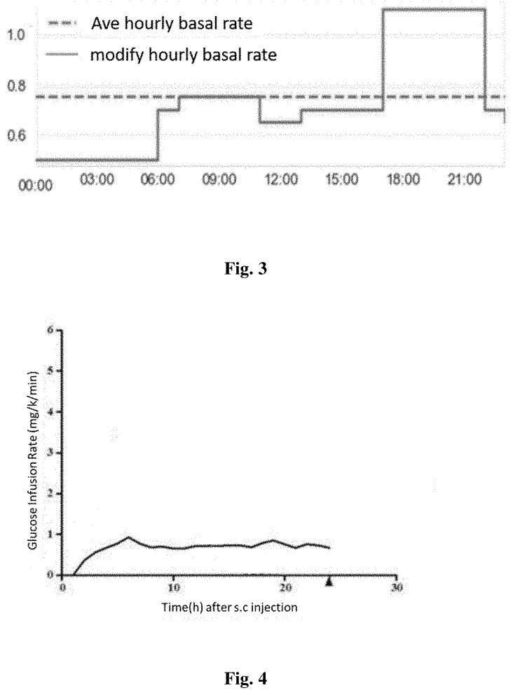

Reference is made to FIG. 3 illustrating an average hourly basal rate (in dashed line) and a modified hourly basal rate pattern calculated by using the teachings of the present invention, taking into account the glucose pattern. For determining the injection time, the control unit determines the optimal correlation between the modified hourly basal rate and the insulin infusion rate. The insulin infusion rate refers to the rate at which the insulin reaches its therapeutic level until its decay. The insulin infusion rate can vary between different insulin types. For example, for FIG. 3, the optimal injection time of Glargine (a type of long acting insulin) would be 3 PM. FIG. 4 illustrates the amount of glucose infused to maintain constant plasma glucose levels and according to which the time of injection is obtained.

In some embodiments, the control unit is configured and operable to determine the active insulin decay time. The active insulin decay time refers to the actual insulin activity time according to the patient glucose and insulin data. The insulin activity time is the time at which insulin stops affecting glucose levels. Usually, this time is defined by the physician arbitrarily, according to the patient's age.

The determination of the active insulin decay time may be implemented by extracting valid bolus events from the input data. It should be noted that the valid bolus may be used to estimate bolus parameters such as required correction factor, carbohydrate ratio, insulin time and insulin decay time. All the following criteria must be met to declare a bolus event as valid: (i) the amount of insulin should be at least a minimal bolus amount, (e.g. the system uses a minimal bolus amount of 0.2 units of insulin, however, this value can be adjusted per patient according to the individual's sensitivity); (ii) if there is carbohydrate intake, the amount of carbohydrate intake should be at least a minimal carbohydrate value (e.g. the minimal carbohydrate value may be about 15 grams of carbohydrates, however, this value can be adjusted per patient according to the individual carbohydrate records); (iii) a glucose level at the time of the bolus should exist; (iv) a glucose data from the time of the bolus until up to 8 hours from the bolus should exist; (v) the timing of the bolus should not exceed a certain threshold from the time of the meal, in case the bolus includes carbohydrate intake (the system may use a special event detection module determining times of extreme increment in the glucose levels that might be caused by carbohydrates intake); (vi) the time of the bolus should be at least at a minimal time interval from a meal time away from another meal event (e.g. the minimal time interval from a meal time may be for example 2 hours); (vii) the time of the bolus should be at least at a minimal time interval from a hypoglycemic time away from a hypoglycemia event (a minimal time interval from a hypoglycemic time may be for example 2 hours).

For the active insulin decay time, the control unit determines for each valid bolus event, the active insulin decay time value by taking into account the glucose pattern (the glucose levels before and after the bolus) and the insulin amount at the time of the bolus (e.g. this may include insulin from preview boluses). The correction to carbohydrate ratio is not mandatory but can enhance the active insulin estimation. The correction to carbohydrates insulin ratio can be taken into account to calculate the ratio between the amount of insulin that aimed to correct the glucose levels, and the amount of insulin that aimed to treat the carbohydrates intake. The post bolus glucose levels, along with the insulin data, help to fit the active insulin time at which the insulin stops to affect the glucose levels for that valid bolus. The most representative active insulin time is then defined as the representative value of all valid events. This active insulin time might also change during the day along with the correction factor and carbohydrate ratio.

1-41. (canceled)

42. A method for use in disease treatment management, the method comprising:
receiving data indicative of pump treatment parameters;
analyzing physiological data during use of a pump; said physiological data being indicative of a physiological characteristic of the patient;
analyzing the received pump treatment parameters data to thereby identify at least one patient-related treatment characteristic; and
creating data indicative of multiple daily injections (MDI) treatment parameters by automatically determining individualized insulin dosing injection parameters data based on said at least one patient-related treatment characteristic or said physiological data.

43. The method of claim 42, wherein said physiological data comprises at least one of: insulin delivery, glucose monitoring data, insulin activity, physical activity, meal event, food type, age, or a metabolic state influencing insulin sensitivity.

44. The method of claim 43, wherein said metabolic state comprises at least one of stress, illness, menstrual cycle, hormonal changes, or drug consumption.

45. The method of claim 42, wherein said at least one patient-related treatment characteristic comprises at least one of insulin response, glucose pattern, meal requirement, or personal metabolic profile or insulin requirements including at least one of total daily insulin requirement or differences in insulin requirements during the course of the day.

46. The method of claim 42, wherein said individualized insulin dosing injection parameters data comprise at least one of: insulin treatment plan including initial insulin dosing parameters, long acting insulin dose, number of required doses of basal insulin, time for basal insulin injection, different types of short acting insulin for different times of the day, short acting insulin dose for meals, Carbohydrate ratio (CR) according to the time of day or correction factor (CF) or individual active time (AI) according to the time of day; or a short-acting insulin dosage component taken according to a sliding scale.

47. The method of claim 45, wherein automatically determining long acting insulin dose comprises analyzing the glucose pattern to define a certain time and a certain number of required doses of the basal insulin.

48. The method of claim 47, wherein said certain time is defined as a time of injecting the long acting insulin dose.

49. The method of claim 45, wherein said analyzing of the glucose pattern comprises identifying at least one pattern of glucose of different levels, or glucose trend as compared to a patient target glucose level.

50. The method of claim 49, wherein, when a plurality of patterns of glucose of different levels or glucose trend is identified, automatically determining long acting insulin dose comprises splitting the long acting insulin injection accordingly and determining times of injections accordingly.

51. The method of claim 42, further comprising at least one of analyzing the received pump treatment parameters data and said physiological data to thereby optimize pump treatment parameters data; converting said individualized insulin dosing injection parameters data from a specific amount to a sliding scale and vice versa; or determining an active insulin decay time.

52. A method for use in disease treatment management, the method comprising:
receiving data indicative of multiple daily injections (MDI) treatment parameters;
analyzing physiological data during multiple daily injections; said physiological data being indicative of a physiological characteristic of the patient;
analyzing said received MDI treatment parameters to thereby identify insulin requirements; and
creating data indicative of pump treatment parameters by automatically determining individualized insulin dosing pump parameters based on said insulin requirements and said physiological data.

53. The method of claim 52, wherein said physiological data comprises at least one of: insulin delivery, glucose monitoring data, insulin activity, physical activity, meal event, food type, age, or a metabolic state influencing insulin sensitivity.

54. The method of claim 53, wherein said metabolic state comprises at least one of stress, illness, menstrual cycle, hormonal changes, or drug consumption.

55. The method of claim 52, wherein said individualized insulin dosing pump parameters include at least one of: daily insulin basal rate including basal intervals and dose; carbohydrate ratio (CR) according to the time of day; correction factor (CF) according to the time of day; individual active insulin time (AI) according to the time of day; or at least one individual glucose target according to the time of day.

56. The method of claim 52, wherein said insulin requirements include at least one of total daily insulin requirement, differences in insulin requirements during the course of the day, glucose patterns, or meal requirements.

57. The method of claim 52, wherein said data indicative of pump treatment parameters comprises total daily basal dose and long acting insulin type.

58. The method of claim 55, wherein automatically determining daily insulin basal rate further comprises at least one of finding a pattern in patient glucose levels and dividing a day into (N) time periods; or defining at least one basal pattern being indicative of hourly basal rate at each daily period.

59. The method of claim 58, further comprising at least one of calculating the carbohydrate ratio, dividing a day into (M) time periods, and changing the carbohydrate ratio proportionally to changes in basal patterns; calculating the correction factor and dividing a day into K time periods, and changing the correction factor proportionally to changes in basal patterns; calculating individual active insulin time based on said physiological data and said data indicative of MDI treatment parameters and dividing a day into L time periods; generating individualized insulin dosing pump parameters configured for at least one of the following: for operating a pump, or for presentation on a user interface; analyzing said received MDI treatment parameters and said physiological data to thereby optimize said received MDI treatment parameters; converting said individualized insulin dosing injection parameters data from a specific amount to a sliding scale and vice versa; or determining an active insulin decay time.

60. The method of claim 52, wherein said MDI treatment parameters are received from at least one of a multiple daily injections (MDI) device, a patient, personal medical data, or a medical practitioner.

61. A control unit for use in disease treatment management, the control unit comprising: a data processor utility configured and operable as an advisor utility for carrying out the method of claim 42.

62. A system for use in disease treatment management, the system comprising:
a control unit configured for receiving physiological data being indicative of a physiological characteristic of a patient and one of the following: data indicative of pump treatment parameters or data indicative of multiple daily injections (MDI) treatment parameters; wherein in a first operative mode said control unit processing utility is configured for processing said data indicative of the pump treatment parameters to thereby create data indicative of MDI injection treatment parameters and in a second operative mode, said control unit processing utility is configured for processing said data indicative of the MDI treatment parameters to thereby create data indicative of pump treatment parameters.

63. The system of claim 62, wherein said control unit processing utility is configured and operable for at least one of (i) identifying at least one of insulin response, glucose pattern, meal requirement, personal metabolic profile or insulin requirements including at least one of total daily insulin requirement or differences in insulin requirements during the course of the day; (ii) converting individualized insulin dosing injection parameters data from a specific amount to a sliding scale and vice versa; (iii) determining an active insulin decay time; or (iv) determining a long acting insulin dose by analyzing a glucose pattern to define a certain time and a certain number of required doses of basal insulin.

64. The system of claim 62, further comprising at least one of a data output utility configured and operable to provide recommendation data regarding the setting of the pump or of the multiple daily injections device; or a memory utility for storing said physiological data and/or said data indicative of pump treatment parameters obtained over a certain time and/or said data indicative of MDI treatment parameters obtained over a certain time.

65. The system of claim 64, wherein when said control unit comprises a data input utility being configured for receiving data indicative of multiple daily injections (MDI) treatment parameters, said recommendation data comprises at least one of insulin dosing pump parameters including at least one of: daily insulin basal rate including basal intervals and dose; at least one carbohydrate ratio (CR) or correction factor (CF) according to the time of day; at least one individual insulin activity time (AI) according to the time of day or at least one glucose target value according to the time of day; wherein calculation of said recommendation data is based on said data indicative of multiple daily injections (MDI) treatment parameters.

66. The system of claim 65, wherein when said data input utility receives data indicative of pump treatment parameters; said recommendation data comprises at least one of insulin treatment plan including initial insulin dosing parameters; long acting insulin dose, number of required doses of basal insulin, time for basal insulin injection, different types of short acting insulin for different times of the day; short acting insulin dose for meals or correction including Carbohydrate ratio (CR) or correction factor (CF) according to the time of day or at least one glucose target value according to the time of day.

67. A computer program recordable on a storage medium; said computer program comprising a machine readable format, the computer program being configured and operable, when being accessed, to carry out the following: receiving and processing physiological data being indicative of a physiological characteristic of the patient and data indicative of multiple daily injections (MDI) treatment parameters or data indicative of pump treatment parameters to thereby create in a first operative mode data indicative of MDI injection treatment parameters for MDI users, and, in a second operative mode, data indicative of pump treatment parameters for pump users.

68. A computer program product, comprising a non-transitory tangible computer readable medium having computer readable program code embodied therein, said computer readable program code adapted to be executed to implement a method as claimed in claim 42.
